(19) United States
(12) Patent Application Publication
(10) Pub. No.: US 2007/0179172 A1
(43) Pub. Date: Aug. 2, 2007

(54) Positive modulators of nicotinic acetylcholine receptors

(76) Inventors: Christopher Becker, Wilmington, DE (US); Jeanne Comstock, West Boylston, MA (US); William F. Michne, Wilmington, DE (US); Megan Murphy, Wilmington, DE (US); Eifion Phillips, Wilmington, DE (US); James D. Rosamond, Wilmington, DE (US); Thomas R. Simpson, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/553,915

(22) PCT Filed: May 4, 2004
(86) PCT No.: PCT/GB04/01934

§ 371 Date: Jul. 13, 2006

(30) Foreign Application Priority Data
May 6, 2003 (SE) 0301320-8

(51) Int. Cl. A61K 31/4743; A61K 31/4741; A61K 31/473

(52) U.S. Cl. 514/290; 514/291

(57) ABSTRACT

Compounds of Formula I or Formula II 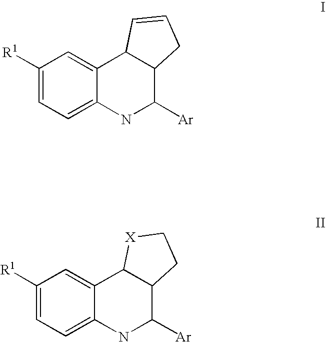
wherein R¹, X and Ar are as described in the specification, pharmaceutically-acceptable salts thereof, processes for preparing them, pharmaceutical compositions containing them and their use in therapy, especially for treatment of conditions associated with reductions in nicotinic transmission.

### TECHNICAL FIELD

The present invention relates to compounds or pharmaceutically-acceptable salts thereof, processes for preparing them, pharmaceutical compositions containing them and their use in therapy. The invention particularly relates to positive modulators of nicotinic acetylcholine receptors, such positive modulator having the capability to increase the efficacy of nicotinic receptor agonists.

### BACKGROUND OF THE INVENTION

Cholinergic receptors normally bind the endogenous neurotransmitter acetylcholine (ACh), thereby triggering the opening of ion channels. ACh receptors in the mammalian central nervous system can be divided into muscarinic (mAChR) and nicotinic (nAChR) subtypes based on the agonist activities of muscarine and nicotine, respectively. The nicotinic acetylcholine receptors are ligand-gated ion-channels containing five subunits. Members of the nAChR subunit gene family have been divided into two groups based on their amino acid sequences; one group containing so-called β subunits, and a second group containing α subunits. Three kinds of α subunits, α7, α8 and α9, have been shown to form functional receptors when expressed alone and thus are presumed to form homooligomeric pentameric receptors.

An allosteric transition state model of the nAChR has been developed taht involves at least a resting state, an activated state and a “desensitized” closed channel state, a process by which receptors become insensitive to the agonist. Different nAChR ligands can stabilize the confonrmational state of a receptor to which they preferentially bind. For example, the agonists ACh and (−)-nicotine respectively stabilize the active and desensitized states.

Changes of the activity of nicotinic receptors has been implicated in a number of diseases. Some of these, for example myasthenia gravis and ADNFLE (autosomal dominant nocturnal front lobe epilepsy) are associated with reductions in the activity of nicotinic transmission either because of a decrease in receptor number or increased desensitization. Reductions in nicotinic receptors have also been hypothesized to mediate cognitive deficits seen in diseases such as Alzheimer's disease and schizophrenia.

The effects of nicotine from tobacco are also mediated by nicotinic receptors, and since the effect of nicotine is to stabilize receptors in a desensitized state, an increased activity of nicotinic receptors may reduce the desire to smoke.

Compounds which bind nACHrs have been suggested for the treatment of a range of disorders involving reduced cholinergic function such as Alzheimer's disease, cognitive or attention disorders, attention deficit hyperactivity disorders, anxiety, depression, smoking cessation, neuroprotection, schizophrenia, analgesia, Tourette's syndrome, and Parkinson's disease.

However, treatment with nicotinic receptor agonists which act at the same site as ACh is problematic because ACh not only activates, but also blocks receptor activity through processes which include desensitization and uncompetitive blockade. Furthermore, prolonged activation appears to induce a long-lasting inactivation. Therefore, agonists of ACh can be expected to reduce activity as well as enhance it.

At nicotinic receptors in general, and of particular note at the α7-nicotinic receptor, desensitization limits the duration of action of an applied agonist.

### DESCRIPTION OF THE INVENTION

We have surprisingly found that certain compounds can increase the efficacy of agonists at nicotinic acetylcholine receptors (nAChR). Compounds having this type of action (hereinafter referred to as “positive modulators”) are likely to be particularly useful for treatment of conditions associated with reductions in nicotinic transmission. In a therapeutic setting such compounds could restore normal interneuronal communication without affecting the temporal profile of activation. In addition, positive modulators are not expected to produce long-term inactivation of receptors as may the prolonged application of agonists.

Positive nAChR modulators of the present invention useful for treatment or prophylaxis of psychotic disorders, intellectual impairment disorders or diseases or conditions in which modulation of the α7 nicotinic receptor is beneficial are compounds in accord with Formula I or Formula II: 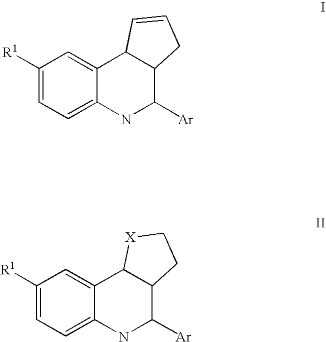
wherein:

R¹ is —OH, —N(R²)₂, —NR²—SO₂—R², —SO₂—N(R²)₂, —CON(R²)₂, or —NR²COR² where R² at each occurrence is independently selected from hydrogen, C₁₋₄alkyl, halogenatedC₁₋₄alkyl, aryl or heteroaryl where any alkyl, halogenated-alkyl, aryl or heteroaryl moiety is substituted with 0, 1, 2 or 3 R³ moieties;

X is O, S or CH₂;

Ar is a moiety selected from furyl, pyridyl, thienyl, phenyl or naphthyl, said moiety having 0, 1, 2, 3 or more R³ substituents where R³ is at each occurrence independently selected from hydrogen, halogen, C₁₋₄alkyl, C₂₋₄alkenyl, C₂₋₄alkynyl, OC₁₋₄alkyl, NH₂, CO₂H, CO₂C₁₋₄alkyl, CN, NO₂, and CF₃;

or a diastereoisomer, enantiomer or pharmaceutically-acceptable salt thereof

Particularly compounds of the inventions are those wherein

R¹ is —SO₂—N(R²)₂ where R² at each occurrence is independently selected from hydrogen, C₁₋₄alkyl, halogenatedC₁₋₄alkyl, aryl or heteroaryl where any alkyl, halogenated-alkyl, aryl or heteroaryl moiety is substituted with 0, 1, 2 or 3 R³ moieties;

X is O, S or CH₂;

Ar is a moiety selected from furyl, pyridyl, thienyl, phenyl or naphthyl, said moiety having 0, 1, 2, 3 or more R³ substituents where R³ is at each occurrence independently selected from hydrogen, halogen, C₁₋₄alkyl, C₂₋₄alkenyl, C₂₋₄alkynyl, OC₁₋₄alkyl, NH₂, CO₂H, CO₂C₁₋₄alkyl, CN, NO₂, and CF₃.

We have also found that 8-hydroxy-4-aryl-substituted 3a,4,5,9b-tetrahydro-3H-cyclopenta[c]quinolines and 8-amino-4-aryl-substituted 3a,4,5,9b-tetrahydro-3H-cyclopenta[c]quinolines are effective positive modulators which can increase the efficacy of agonists at nicotinic receptors and which therefore can be used in the methods of the invention.

Thus, in one aspect the invention is a method of treatment or prophylaxis of psychotic disorders, intellectual impairment disorders or diseases or conditions in which modulation of the α7 nicotinic receptor is beneficial, which method comprises administering a therapeutically-effective amount of a positive modulator of Formula I or formula II as described above

or a diastereoisomer, enantiomer or pharmaceutically-acceptable salt thereof.

A particular aspect of the method of the invention is a method of treatment for Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, ulcerative colitis or irritable bowel syndrome.

Methods of treatment of this invention include administering either a positive modulator as the only active substance, thus modulating the activity of endogenous nicotinic receptor agonists such as acetylcholine or cholines, or administering a positive modulator together with a nicotinic receptor agonist.

In a particular form of this aspect of the invention, the method of treatment comprises treatment with an α7-nicotinic receptor modulator as described herein and an α7-nicotinic receptor agonist. An example of a suitable α7-nicotinic receptor agonist is (−)-spiro[1-azabicyclo[2.2.2.]octane-3,5′-oxazolidine]-2′-one. Other α7-nicotinic receptor agonists useful for treatment in conjunction with positive modulators of the present invention are described in international publications WO 96/06098, WO 97/30998 and WO 99/03859.

In another aspect the invention is compounds in accord with Formula I or Formula II 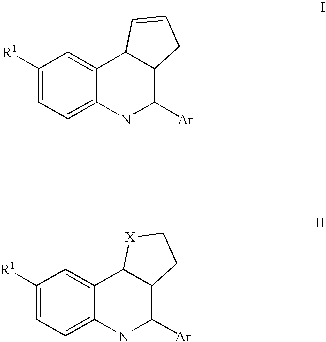
wherein:

R¹ is NR²—SO₂—R² or —SO₂—N(R²)₂ where R² at each occurrence is independently selected from hydrogen, C₁₋₄alkyl, halogenatedC₁₋₄alkyl, aryl or heteroaryl where any alkyl, halogenated-alkyl, aryl or heteroaryl moiety is substituted with 0, 1, 2 or 3 R³ moieties;

X is O, S or CH₂;

Ar is a moiety selected from furyl, pyridyl, thienyl, phenyl or naphthyl, said moiety having 0, 1, 2, 3 or more R³ substituents where R³ is at each occurrence independently selected from hydrogen, halogen, C₁₋₄alkyl, C₂₋₄alkenyl, C₂₋₄alkynyl, OC₁₋₄alkyl, NH₂, CO₂H, CO₂C₁₋₄alayl, CN, NO₂, and CF₃;

or a diastereoisomer, enantiomer or pharmaceutically-acceptable salt thereof.

More particular compounds of the invention include:
4-(2-methylphenyl)-3a,4,5,9b-tetrahydro-3H-cyclopenta[c]quinoline-8-sulfonamide;
4-(4-methylphenyl)-3a,4,5,9b-tetrahydro-3H-cyclopenta[c]quinoline-8-sulfonamide;
4-(3,4,5-trimethoxyphenyl)-3a,4,5,9b-tetrahydro-3H-cyclopenta[c]quinoline-8-sulfonamide;
4-(2-methyl-4,5-dimethoxyphenyl)-3a,4,5,9b-tetrahydro-3H-cyclopenta[c]quinoline-8-sulfonamide;
4-(3,5-dimethoxyphenyl)-3a,4,5,9b-tetrahydro-3H-cyclopenta[c]quinoline-8-sulfonamide;
4-(4-tert-butylphenyl)-3a,4,5,9b-tetrahydro-3H-cyclopenta[c]quinoline-8-sulfonamide;
4-(2-naphthyl)-3a,4,5,9b-tetrahydro-3H-cyclopenta[c]quinoline-8-sulfonamide;
4-(4-fluorophenyl)-3a,4,5,9b-tetrahydro-3H-cyclopenta[c]quinoline-8-sulfonamide;
4-(4-methylphenyl)-2,3,3a,4,5,9b-hexahydro-furo[3,2-c]quinoline-8-sulphonamide;
(3aR,4S,9bS)-4-naphthalen-2-yl-3a,4,5,9b-tetrahydro-3H-cyclopenta[c]quinoline-8-sulphonamide,
(3aS,4R,9bR)-4-naphthalen-2-yl-3a,4,5,9b-tetrahydro-3H-cyclopenta[c]quinoline-8-sulphonamide,
(3aR,4S,9bS)-4-(4-methylphenyl)-3a,4,5,9b-tetrahydro-3H-cyclopenta[c]quinoline-8-sulfonamide;
(3aS,4R,9bR)-4-(4-methylphenyl)-3a,4,5,9b-tetrahydro-3H-cyclopenta[c]quinoline-8-sulfonamide;
(3aS,4S,9bR)-4-(4-methylphenyl)-3a ,4,5,9b-tetrahydro-3H-cyclopenta[c]quinoline-8-sulfonamide;
(3aR,4R,9bS)-4-(4-methylphenyl)-3a,4,5,9b-tetrahydro-3H-cyclopenta[c]quinoline-8-sulfonamide;
(3aR,4S,9bS)-4-(4-methylphenyl)-1,2,3a,4,5,9b-hexahydro-3H-cyclopenta[c]quinoline-8-sulfonamide, and
(3aS,4R,9bR)-4-(4-methylphenyl)-1,2,3a,4,5,9b-hexahydro-3H-cyclopenta[c]quinoline-8-sulfonamide

or a pharmaceutically-acceptable salt thereof

Another aspect of the invention comprises methods of preparing compounds according to Formula I or Formula II. In what follows, unless otherwise indicated, R¹ and Ar are as defined herein for Formula I and Formula II.

Compounds of Formula I or Formula II may be prepared, for example, as outlined in Scheme 1, via a 3-component coupling reaction of a suitably subsituted aromatic amine of formula II, aromatic aldehyde of formula III and alkene of formula IV. The reaction may be performed in the presence of a suitable acidic catalyst, for example a protic acid such as trifluoroacetic acid, or a suitable Lewis Acid catalyst, such as indium trichloride, a drying agent such as molecular sieves, in a solvent such as acetonitrile. Compounds of formula II, III, and IV are commercially available, or may be prepared by methods described in the literature, or may be prepared using methods and techniques known to persons skilled in the art of organic chemistry synthesis.

Positive modulators of the invention have the advantage that they are less toxic, more efficacious, longer acting, have a broader range of activity, be more potent, produce fewer side effects, are more easily absorbed or have other useful pharmacological properties.

Acid addition salts re also within the scope of the invention. Such salts include salts of mineral acids, for example the hydrochloride and hydrobromide salts; and salts formed with organic acids such as formate, acetate, maleate, benzoate, tartrate, and fumarate salts. Acid addition salts of compounds of Formula I or Formula II may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

The compounds of Formula I and Formula II may exist in tautomeric or enantiomeric forms, all of which are included within the scope of the invention. The various optical isomers may be isolated by separation of a racemic mixture of the compounds using conventional techniques, for example by fractional crystallization, or chiral HPLC. Alternatively the individual enantiomers may be made by reaction of the appropriate optically active starting materials under reaction conditions which will not cause racemization.

A further aspect of the invention comprises a pharmaceutical composition for treating or preventing a condition or disorder as described herein arising from dysfunction of nicotinic acetylcholine receptor neurotransmission in a mammal, preferably a human. Such a pharmaceutical composition comprises a therapeutically-effective amount of a compound of Formula I or Formula II, an enantiomer thereof or a pharnaceutically-acceptable salt thereof, effective in treating or preventing such disorder or condition and a pharmaceutically-acceptable carrier.

Another aspect of the invention is a pharmaceutical composition comprising a compound according to Formula I or Formula II as described herein or a diastereoisomer, enantiomer or pharmaceutically-acceptable salt thereof, together with at least one pharmaceutically-acceptable diluent or carrier.

In particular, this aspect of the invention provides a pharmaceutical composition including preferably less than 80% and more preferably less than 50% by weight of a compound of the invention in admixture with a pharmaceutically-acceptable diluent or carrier.

Examples of diluent and carriers are:

for tablets and dragees: lactose, starch, talc, stearic acid;

for capsules: tartaric acid or lactose;

for injectable solutions: water, alcohols, glycerin, vegetable oils;

for suppositories: natural or hardened oils or waxes.

Yet another pharmaceutical composition of the invention comprises in addition a nicotinic receptor agonist.

Another aspect of the invention provides a process for the preparation of a pharmaceutical composition, which comprises incorporating the ingredients in a composition by conventional processes.

Yet a further aspect of the invention is the use of a compound according to Formula I or Formula II, an enantiomer thereof or a pharmaceutically-acceptable salt thereof, for the preparation of a medicament.

A particular aspect of the invention is the use of a compound according to Formula I or Formula II as described herein or a diastereoisomer, enantiomer or pharmaceutically-acceptable salt thereof, in the manufacture of a medicament for the treatment or prophylaxis of psychotic disorders, intellectual impairment disorders, human diseases or conditions in which modulation of the α7 nicotinic receptor is beneficial including Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, ulcerative colitis or irritable bowel syndrome.

In a particular form, this aspect of the invention is the use of compound according to the invention in the manufacture of a medicament for tie treatment or prophylaxis of a condition associated with reduced nicotinic receptor transmission or a condition associated with reduced nicotinic receptor density which could be one of the diseases or conditions mentioned herein, which treatment comprises administering said medicament comprising a therapeutically effective amount of a compound according to the invention to a patient.

It will be understood that this use includes the manufacture of medicaments comprising either a positive modulator as the only active substance providing modulation of the activity of endogenous nicotinic receptor agonists, or the manufacture of medicaments comprising a positive modulator in combination with a nicotinic receptor agonist. Thus, this use provides for the manufacture of medicaments containing a positive modulator and medicaments containing in addition a nicotinic receptor agonist.

In a particular form of this aspect of the invention, the medicament or pharmaceutical composition comprises an α7-nicotinic receptor modulator as described herein and an α7-nicotinic receptor agonist. An example of a suitable α7-nicotinic receptor agonist is (−)-spiro[1-azabicyclo[2.2.2.]octane-3,5′-oxazolidine]-2′-one. Other α7-nicotinic receptor agonists useful in medicaments in conjunction with positive modulators of the present invention are described in international publications WO 96/06098, WO 97/30998 and WO 99/03859.

Still a further aspect of the invention is a method of treating or preventing a condition or disorder in mammals and particularly humans as mentioned herein arising from dysfunction of nicotinic acetylcholine receptor neurotransmission.

A particular form of this aspect of the invention provides a method for the treatment of a condition associated with reduced nicotine transmission, by administering to a patient in need of such treatment, a medically effective amount of a positive modulator of a nicotinic receptor agonist, said positive modulator having the capability to increase the efficacy of the said nicotinic receptor agonist.

In the above-mentioned compositions, uses and methods, the amount of a compound according to Formula I or Formula II employed will, of course, vary with the compound employed, the mode of administration and the treatment desired. However, in general, satisfactory results will be obtained when a compound of the invention is administered to provide a daily dosage of from about 0.1 mg to about 20 mg per kg of animal body weight, which may be given as divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carrier or diluent.

In compositions, uses and methods of the invention, a compound of Formula I or Formula II, an enantiomer thereof, or a pharmaceutically-acceptable salts thereof, may be used on its own in the form of appropriate medicinal preparations for enteral or parenteral administration or may be used in a composition containing other pharmacologically-active agents. For example, a composition containing other pharmacologically-active agents may contain a positive modulator compound according to Formula I or Formula II together with a nicotinic receptor agonist.

Accordingly, the invention includes compositions comprising a positive modulator as the only active substance, thus modulating the activity of endogenous nicotinic receptor agonists such as acetylcholine or cholines, and compositions comprising a positive modulator in combination with a nicotinic receptor agonist. Thus, the said pharmaceutical compositions containing a positive modulator of a nicotinic receptor agonist may, in addition, comprise a nicotinic receptor agonist.

Examples of diseases or conditions for which aspects of the present invention are useful include schizophrenia, mania and manic depression, anxiety, Alzheimer's disease, learning deficit, cognition deficit, attention deficit, memory loss, Lewy Body Dementia, Attention Deficit Hyperactivity Disorder, Parkinson's disease, Huntington's disease, Tourette's syndrome, jetlag, and nicotine addiction (including that resulting from exposure to products containing nicotine).

It will be understood that the a positive modulator of the invention can be administered either with the purpose of modulating the action of endogenous nicotine receptor agonists such as acetylcholine or cholines, or to modulate the action of an exogenous nicotinic receptor agonist.

Experimental Methods

The activity of the compounds of the invention may be measured in the tests set out below:

(a) Xenopus oocyte Current Recording

The Xenopus oocyte has provided a powerful means of assessing the function of proteins thought to be subunits of ligand-gated ion-channels. Injection of RNA transcribed from cDNA clones encoding the appropriate receptor subunits, or injection of cDNA in which the coding sequence is placed downstream of a promoter, results in the appearance of functional ligand-gated ion-channels on the surface of the oocyte (see e.g. Boulter et al. (1987) Proc. Natl. Acad. Sci. U.S.A. 84, 7763-7767).

Consequently, one convenient technique to assess the enhancement of nicotinic efficacy is two-electrode voltage-clamp recording from Xenopus oocytes expressing α7-nicotinic receptors from cRNA.

Xenopus laevis frogs (Xenopus I, Kalamazoo, Mich.) were anesthetized using 0.15% tricaine. Oocytes were removed to OR2 solution (82 mM NaCl, 2.5 mM KCl, 5 mM HEPES, 1.5 mM NaH₂PO₄, 1 mM MgCl₂, 0.1 mM EDTA; pH 7.4). The oocytes were defolliculated by incubation in 25 ml OR2 containing 0.2% collagenase 1A (Sigma) two times for 60 min on a platform vibrating at 1 Hz and stored in Leibovitz's L-15 medium (50 μg/ml gentomycin, 10 Units/ml penicillin, and 10 μg/ml streptomycin). Approximately 50 ng of cRNA was injected in each oocyte the following day. cRNA was synthesised from cDNA using Message Machine (purchased from Abion).

The external recording solution consisted of 90 mM NaCl, 1 mM KCl, 1 mM MgCl₂, 1 mM BaCl₂, 5 mM HEPES; pH 7.4. Two-electrode voltage-clamp recording was carried out using an Oocyte Clamp amplifier (OC 725C; Warner Instrument, Hamden, Conn.). Oocytes were impaled with two electrodes of 1-2 MΩ tip resistance when filled with 3M KCl. Recordings were begun when membrane potential became stable at potentials negative to −20 mV (resting membrane potentials are less negative when Ba⁺⁺ replaces Ca⁺⁺ in bathing solutions). Membrane potential was clamped at −80 mV. ACh was purchased from Sigma. Oocytes were continuously perfused (5 ml/min) with recording solution with or without ACh.

Current amplitude was measured from baseline to peak. EC50 values, maximal effect, and Hill slopes were estimated by fitting the data to the logistic equation using GraphPad Prism (GraphPad Software, Inc., San Diego, Calif.).

Increases in agonist efficacy elicited by a positive modulator can be calculated in two ways:

(1) As percent potentiation of current amplitude which is defined as 100(Im−Ic)/Ic where Im is current amplitude in the presence of modulator and Ic is current in the absence of modulator.

(2) As percent potentiation of “area under curve” of an agonist trace, which is the integration of net current over time. Area under the curve is a common representation of the total ion flux through the channel.

(b) Ca⁺⁺ Flux Imaging

Imaging of Ca⁺⁺ flux through nAChR α7 receptors transiently expressed in a cell line is another means of assaying modulator activity.

Cells expressing α7 receptors (for example HEK-293 cells or cell cultured neurons) are grown to confluence in 96 well plates and loaded with fluo-3, a fluorescent calcium indicator. To screen for α7 modulatory activity, the 96 well plate is placed in a fluorescence imaging plate reader (FLIPR) and test compounds along with an α7 agonist are applied simultaneously to all wells. Receptor activation is measured by calcium influx into cells which is quantified by the increase in fluorescence intensity of each well, recorded simultaneously by the FLIPR. A modulatory effect is determined by the increase in fluorescence over that of agonist alone. Similarly, to test for nAChR α7 agonist activity, test compounds along with an α7 modulator are applied simultaneously to all wells. Receptor activation is measured by calcium influx into cells which is quantified by the increase in fluorescence intensity of each well, recorded simultaneously by the FLIPR. An agonist effect is determined by the increase in fluorescence over that of modulator alone.

Cell-cultured neurons are prepared according to the following method: Eighteen day old Sprague-Dawley rat fetuses (E-18) were aseptically removed from the pregnant female, sacrificed, the frontal cortices of the brains removed, the meninges stripped, and the cleaned cortex placed into cold HBSS. If hippocampus was desired, the hippocampus was dissected away from the cortex and then placed into cold HBSS. The tissues were mechanically dispersed, washed once in HBSS (200 g for 30 min in 4° C.) resuspended in a modification of Sato's medium supplemented with glutamine, antibiotics, potassium chloride, insulin, transferrin, selenium, and 5% heat-inactivated fetal bovine serum (PBS; endotoxin free) and plated into each of a 24-well plate (coated with poly-L-lysine). The wells could contain glass cover slips which were also coated with PLL. The plates were incubated at 37° C. in a CO₂ incubator. After 24 hours the medium was removed, fresh medium added, and the cells allowed to grow for at least another 11 days, feeding when necessary.

The compounds of the invention are compounds, which causes a 100% potentiation (2-fold increase) of baseline current (as described above), as measured baseline to peak at low concentration of acetylcholine (30 μM), indicating that they are expected to have useful therapeutic activity. The compounds of the invention are also compounds, which increase the flux of Ca⁺⁺ when applied in the Ca2+ flux-imaging assay, as described above. Any increase of Ca⁺⁺ flux, caused by a compound of the invention, compared to the Ca⁺⁺ flux caused by an agonist alone (as measured in Fluorescence Intensity Units) indicates that they are expected to have useful therapeutic activity.

The use of compounds of the invention have the advantage that they may be less toxic, be more efficacious, be longer acting, have a broader range of activity, be more potent, produce fewer side effects, are more easily absorbed or have other useful pharmacological properties.

General Experimental Procedures

The invention is illustrated by, but not limited to, examples described herein in which descriptions, where applicable and unless otherwise stated, the following terms, abbreviations and conditions are used:

Commercial reagents were used without further purification.

The following abbreviations are used herein: aq., aqueous; atm, atmospheric pressure; BOC, 1,1-dimethylethoxycarbonyl; DCM, dichloromethane; DMF, N,N-dimethylformamide; DMSO, dimethyl sulfoxide; EtOH, ethanol; Et2O, diethyl ether; EtOAc, ethyl acetate; h, hour(s); HPLC, high pressure liquid chromatography; HOBT, 1-hydroxybenzotriazole; MeOH, methanol; min, minutes; MS, mass spectrum; NMR nuclear magnetic resonance; psi, pounds per square inch; RT, room temperature; sat., saturated; TEA, triethylamine; TFA, trifluoroacetic acid; THF, tetrahydrofuran.

Temperatures are given in degrees Celsius (° C.); unless otherwise stated, operations were carried out at room or ambient temperature (18-25° C.).

Organic solutions were dried over anhydrous sodium or magnesium sulfate; evaporation of solvent was carried out using a rotary evaporator under reduced pressure (4.5-30 mm Hg) with a bath temperature of up to 60° C.

Chromatography means flash column chromatography on silica gel unless otherwise noted; solvent mixture compositions are given as volume percentages or volume ratios.

When given, NMR data is in the form of delta values for major diagnostic protons (given in parts per million (ppm) relative to tetramethylsilane as an internal standard) determined at 300 MHz.

Melting points are uncorrected.

Mass spectra were recorded using either a Hewlett Packard 5988A or a MicroMass Quattro-1 Mass Spectrometer and are reported as m/z for the parent molecular ion. Room temperature refers to 20-25° C.

Reactions described herein, unless otherwise noted, are usually conducted at a pressure of about one to about three atmospheres, preferably at ambient pressure (about one atmosphere).

Unless otherwise stated, the reactions are conducted under an inert atmosphere, preferably under a nitrogen atmosphere.

The compounds of the invention and intermediates may be isolated from their reaction mixtures by standard techniques.

As used herein, unless otherwise indicated, “C₁₋₄alakyl” includes methyl, ethyl, n-propyl, n-butyl, i-propyl, i-butyl, t-butyl, s-butyl, and the like, and C₃₋₆alkyl moieties may be straight-chained, branched or cyclic, for example cyclopropyl or cyclobutyl.

As used herein, unless otherwise indicated, “C₂₋₄alkenyl” includes but is not limited to 1-propenyl, 2-propenyl, 1-butenyl, 2-butenyl and 3-butenyl.

As used herein, unless otherwise indicated, “C₂₋₄alkynyl” includes but is not limited to ethynyl, 1-propynyl, 2-propynyl, 1-butynyl, 2-butynyl and 3-butynyl.

As used herein “halogen” means fluoride, chloride, bromide, or iodide.

### EXAMPLES

Compounds of the invention may be made generally by the process illustrated in Scheme 1 wherein R1, Ar and X are as defined herein for compounds of Formula I or II. 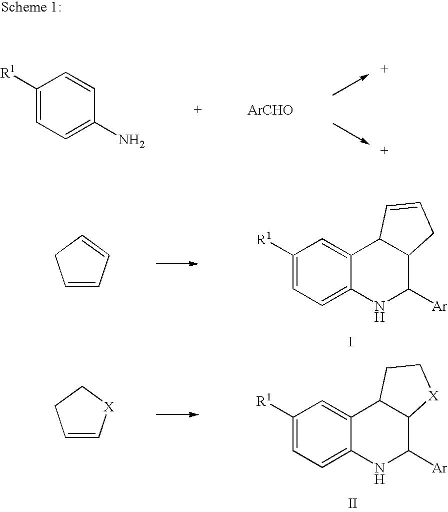
In all processes described herein, where necessary, hydroxy, amino or other reactive groups may be protected using a protecting group as will be understood by those of skill in the art.

The preparation of 4-aryl-3a,4,5,9b-tetrahydro-3H-cyclopenta[c]quinoline-8-sulfonic acid amides or reverse sulfonamides may be generally achieved by the processes illustrated below:

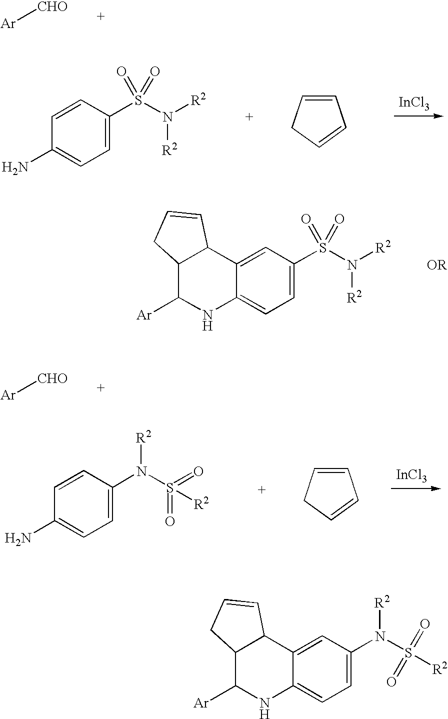

For example, to a solution of an arylaldehyde (3.2 mmol), a 4-aminobenzenesulfonamide (3.2 mmol), and cyclopentadiene (0.63 g, 9.6 mmol) in acetonitrile (10 mL) was added indium trichloride (0.142 g, 6.64 mmol) and the mixture was stirred at rt overnight. Aqueous 10% Na₂CO₃ (10 mL) was added and the product was extracted into chloroform (3×10 mL), washed with water and brine, dried over MgSO₄, and concentrated under reduced pressure. The residue was purified by column chromatography on silica gel and eluted with hexane ethyl acetate and the combined product fractions were freeze dried from a mixture of acetonitrile and water to afford a quinoline.

More specifically, compounds according to Formula I or Formula II as described herein may be prepared by adding indium chloride to a solution of an arylaldehyde, a 4-aminobenzenesulfonamide, and cyclopentadiene or 2,3-dihydrofuran in acetonitrile, stirring overnight then neutralizing, extracting, concentrating and purifying to afford a quinoline.

The following examples may be prepared accordingly by use of the appropriate precursors.

### Example 1

### 4-(1-Naphthyl)-3a,4,5,9b-tetrahydro-3H-cyclopenta[c]quinoline-8-sulfonamide

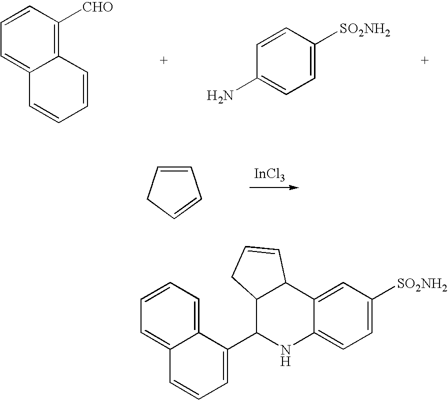

Yield, 0.83 g (69%); ¹HNMR (500 MHz, DMSO-d₆) δ 8.28 (d, 1H), 7.98 (d, 1H), 7.89 (d, 1H), 7.75 (d, 1H), 7.58 (m, 3H), 7.49 (s, 1H), 7.37 (t, 1H), 6.98 (s, 2H), 6.88 (d, 1H), 6.34 (s, 1H), 5.91 (s, 1H), 5.59 (d, 1H), 5.44 (s, 1H), 4.25 (d, 1H), 3.17 (m, 1H), 2.41 (m, 1H), 1.42 (m, 1H); MS (ES+) m/z: 377 (M+1); Anal. Calcd. for C₂₂H₂₀N₂O₂S.¼H₂O: C, 69.36; H, 5.42; N, 7.35. Found: C, 69.29; H, 5.49; N, 7.46.

### Example 2

### 4-(Phenyl)-3a,4,5,9b-tetrahydro-3H-cyclopenta[c]quinoline-8-sulfonamide

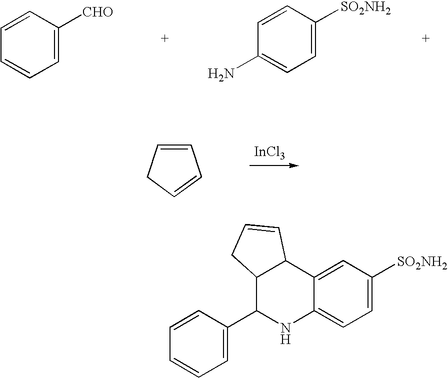

Yield 0.37 g (35%); ¹H NMR (500 MHz, DMSO-d₆) δ 7.46 (m, 3H), 7.39 (m, 2H), 7.31 (m, 2H), 6.95 (s, 2H), 6.81 (d, 1H), 6.37 (s, 1H), 5.89 (d, 1H), 5.62 (d, 1H), 4.64 (s, 1H), 4.07 (d, 1H), 2.95 (m, 1H), 2.39 (m, 1H), 1.64 (m, 1H); MS (ES+) m/z: 327 (M+1); Anal. Calcd. for C₁₈H₁₈N₂O₂S.0.65CH₃CN: C, 65.58; H, 5.70; N, 10.50. Found: C, 65.35; H, 5.73; N, 10.54.

### Example 3

### 4-(2-Nitrophenyl)-3a,4,5,9b-tetrahydro-3H-cyclopenta[c]quinoline-8-sulfonamide

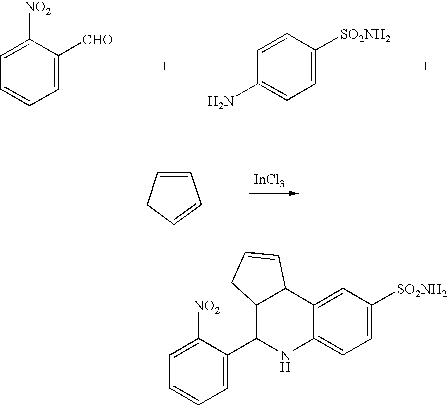

Yield 0.24 g (20%); ¹H NMR (500 MHz, DMSO-d₆) δ 7.97 (m, 1H), 7.92 (m, 1H), 7.80 (m, 1H), 7.60 (m, 1H), 7.47 (s, 1H), 7.36 (m, 1H), 6.98 (s, 2H), 6.78 (d, 1H), 6.37 (s, 1H), 5.94 (m, 1H), 5.67 (m, 1H), 4.96 (m, 1H), 4.09 (m, 1H), 3.09 (m, 1H), 2.55 (m, 1H), 1.70 (m, 1H); MS (ES+) m/z: 372 (M+1).

### Example 4

### 4-(3-Methylphenyl)-3a,4,5,9b-tetrahydro-3H-cyclopenta[c]quinoline-8-sulfonamide

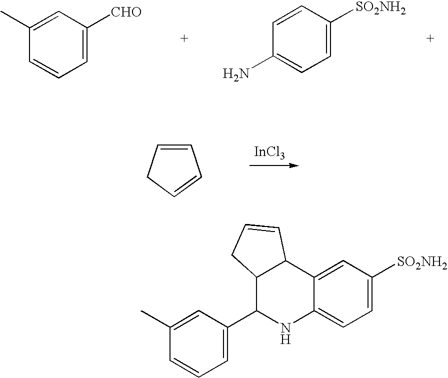

Yield 0.53 g (49%); ¹H NMR (500 MHz, DMSO-d₆) δ 7.42 (s, 1H), 7.35 (d, 1H), 7.32 (m, 3H), 7.11 (d, 1H), 6.94 (s, 2H), 6.81 (d, 1H), 6.34 (s, 1H), 5.88 (d, 1H), 5.62 (d, 1H), 4.59 (d, 1H), 4.05 (m, 1H), 2.93 (m, 1H), 2.40 (m, 1H), 2.37 (s, 3H), 1.65 (m, 1H); MS (ES+) m/z: 341 (M+1); Anal. Calcd. for C₁₉H₂₀N₂O₂S: C, 67.03; H, 5.92; N, 8.23. Found: C, 67.23; H, 5.85; N, 7.95.

### Example 5

### 4-(2-Methylphenyl)-3a,4,5,9b-tetrahydro-3H-cyclopenta[c]quinoline-8-sulfonamide

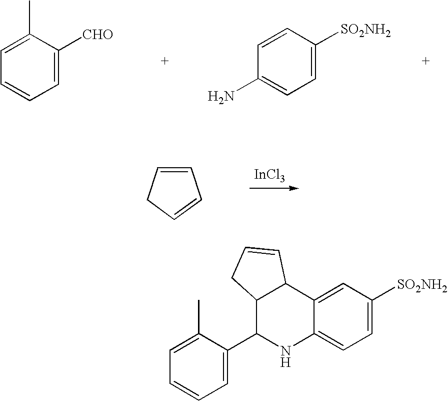

Yield 0.65 g (60%); ¹H NMR (500 MHz, DMSO-d₆) δ 7.51 (d, 1H), 7.44 (s, 1H), 7.32 (m, 1H), 7.24 (m, 1H), 7.58 (m, 2H), 6.94 (s, 2H), 6.80 (d, 1H), 6.21 (s, 1H), 5.89 (s, 1H), 5.63 (d, 1H), 4.79 (d, 1H), 4.10 (d, 1H), 2.98 (m, 1H), 2.45 (m, 1H), 2.37 (s, 3H), 1.60 (m, 1H); MS (ES+) m/z: 341 (M+1); Anal. Calcd. for C₁₉H₂₀N₂O₂S: C, 67.03; H, 5.92; N, 8.22. Found: C, 66.97; H, 6.10; N, 8.15.

### Example 6

### 4-(4-Methylphenyl)-3a,4,5,9b-tetrahydro-3H-cyclopenta[c]quinoline-8-sulfonamide

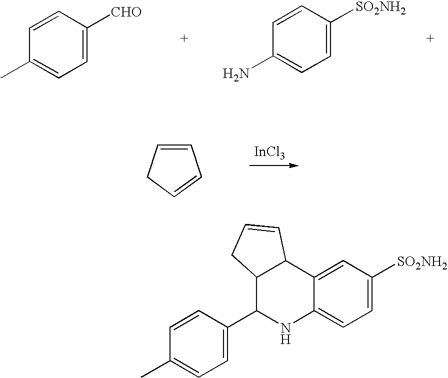

Yield 0.26 g (24%); ¹H NMR (500 MHz, DMSO-d₆) δ 7.43 (s, 1H), 7.32 (m, 3H), 7.20 (m, 2H), 6.94 (s, 2H), 6.80 (d, 1H), 6.31 (s, 1H), 5.88 (s, 1H), 5.62 (d, 1H), 4.59 (d, 1H), 4.06 (m, 1H), 2.92 (m, 1H), 2.38 (m, 1H), 2.31 (s, 3H), 1.65 (m, 1H); MS (ES+) m/z: 341 (M+1); Anal. Calcd. for C₁₉H₂₀N₂O₂S: C, 67.03; H, 5.92; N, 8.22. Found: C, 66.35; H, 5.92; N, 8.29.

### Example 7

### 4-(3,4,5-Trimethoxyphenyl)-3a,4,5,9b-tetrahydro-3H-cyclopenta[c]quinoline-8-sulfonamide

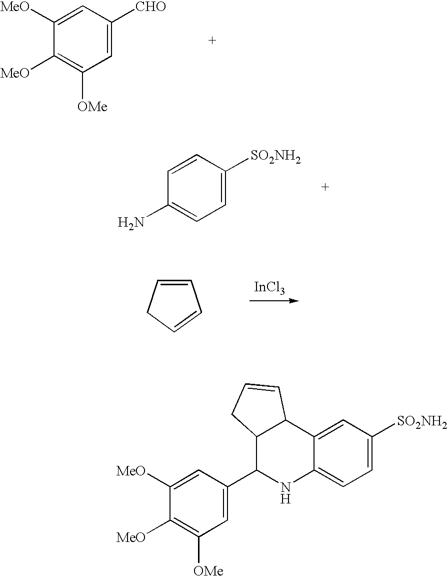

Yield 0.34 g (26%); ¹H NMR (500 MHz, DMSO-d₆) δ 7.43 (s, 1H), 7.33 (m, 1H), 6.95 (s, 2H), 6.80 (d, 1H), 6.72 (m, 2H), 6.31 (s, 1H), 5.89 (m, 1H), 5.64 (m, 1H), 5.55 (m, 1H), 4.05 (m, 1H), 3.80 (s, 6H), 3.66 (s, 3H), 2.96 (m, 1H), 2.42 (m, 1H), 1.73 (m, 1H); MS (ES+) m/z: 417 (M+1); Anal. Calcd. for C₂₁H₂₄N₂O₅S: C, 60.56; H, 5.81; N, 6.72. Found: C, 60.42; H, 5.90; N, 6.46.

### Example 8

### 4-(2-Methyl-4,5-dimethoxyphenyl)-3a,4,5,9b-tetrahydro-3H-cyclopenta[c]quinoline-8-sulfonamide

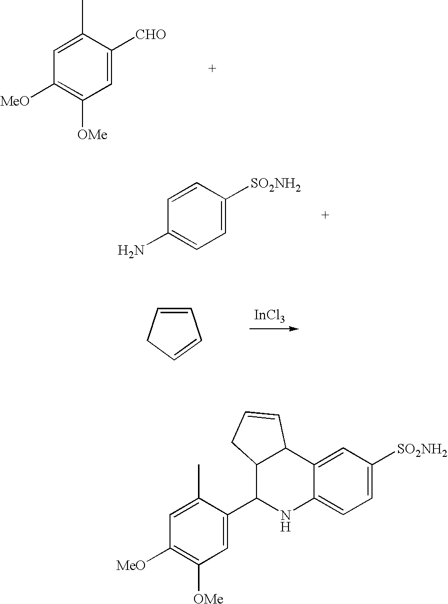

Yield 0.32 g (25%); ¹H NMR (500 MHz, DMSO-d₆) δ 7.43 (s, 1H), 7.32 (m, 1H), 7.25 (s, 1H), 6.93 (s, 2H), 6.77 (m, 2H), 6.14 (s, 1H), 5.86 (m, 1H), 5.63 (m, 1H), 4.69 (m, 1H), 4.06 (m, 1H), 3.78 (s, 3H), 2.91 (m, 1H), 2.47 (m, 1H), 2.33 (s, 3H), 2.15 (s, 3H), 1.64 (m, 1H).

### Example 9

### 4-(3,5-Dimethoxyphenyl)-3a,4,5,9b-tetrahydro-3H-cyclopenta[c]quinoline-8-sulfonamide

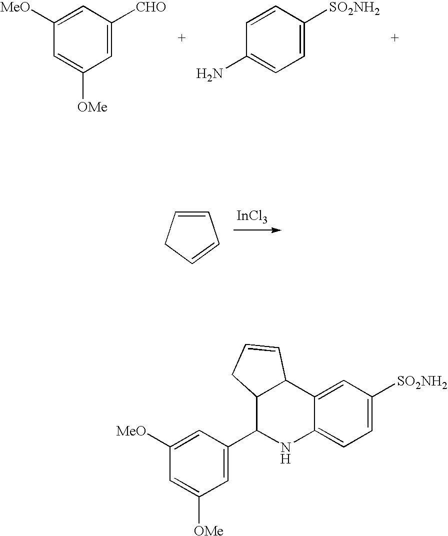

Yield 0.42 g (34%); ¹H NMR (500 MHz, DMSO-d₆) δ 7.42 (s, 1H), 7.33 (d, 1H), 6.94 (s, 2H), 6.81 (d, 1H), 6.61 (s, 2H), 6.42 (s, 1H), 6.32 (s, 1H), 5.88 (m, 1H), 5.62 (m, 1H), 4.55 (m, 1H), 4.05 (m, 1H), 3.76 (d, 6H), 2.97 (m, 1H), 2.36 (m, 1H), 1.70 (m, 1H); MS (ES+) m/z: 387 (M+1); Anal. Calcd. for C₂₀H₂₂N₂O₄S: C, 62.15; H, 5.74; N, 7.25. Found: C, 61.81; H, 5.64; N, 7.32.

### Example 10

### 4-(4-tert-Butylphenyl)-3a,4,5,9b-tetrahydro-3H-cyclopenta[c]quinoline-8-sulfonamide

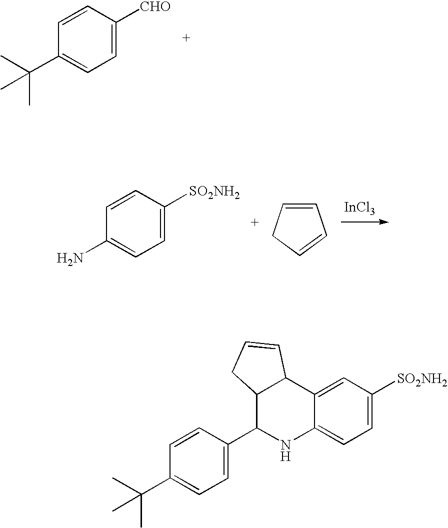

Yield 0.10 g (8%); ¹H NMR (500 MHz, DMSO-d₆) δ 7.36 (m, 6H), 6.93 (s, 2H), 6.77 (d, 1H), 6.32 (s, 1H), 5.88 (m, 1H), 5.63 (m, 1H), 4.58 (d, 1H), 4.06 (m, 1H), 2.92 (m, 1H), 2.43 (m, 1H), 1.70 (m, 1H), 1.30 (s, 9H); MS (ES+) m/z: 383 (M+1); Anal. Calcd. for C₂₂H₂₆N₂O₂S: C, 69.08; H, 6.85; N, 7.32. Found: C, 68.60; H, 6.82; N, 6.83.

### Example 11

### 4-(2-Naphthyl)-3a,4,5,9b-tetrahydro-3H-cyclopenta[c]quinoline-8-sulfonamide

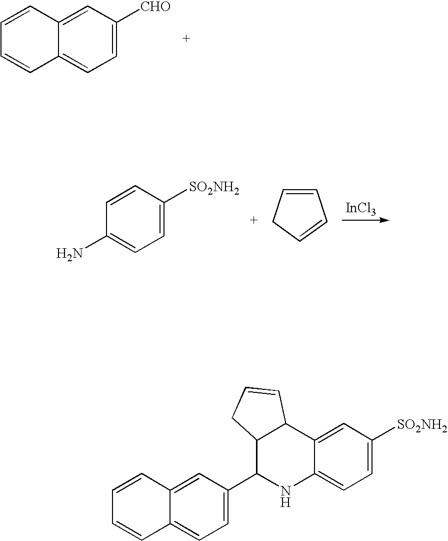

Yield 0.23 g (19%); ¹H NMR (500 MHz, DMSO-d₆) δ 7.96 (m, 4H), 7.63 (m, 1H), 7.52 (m, 2H), 7.47 (s, 1H), 7.36 (m, 1H), 6.97 (s, 2H), 6.87 (d, 1H), 6.52 (s, 1H), 5.91 (d, 1H), 5.61 (d, 1H), 4.81 (d, 1H), 4.12 (d, 1H), 3.08 (m, 1H), 2.45 (m, 1H), 1.61 (m, 1H); MS (ES+) m/z: 377 (M+1); Anal. Calcd. for C₂₂H₂₀N₂O₂S: C, 70.18; H, 5.35; N, 7.44. Found: C, 70.70; H, 5.33; 6.97.

### Example 12

### 4-(4-Fluorophenyl)-3a,4,5,9b-tetrahydro-3H-cyclopenta[c]quinoline-8-sulfonamide

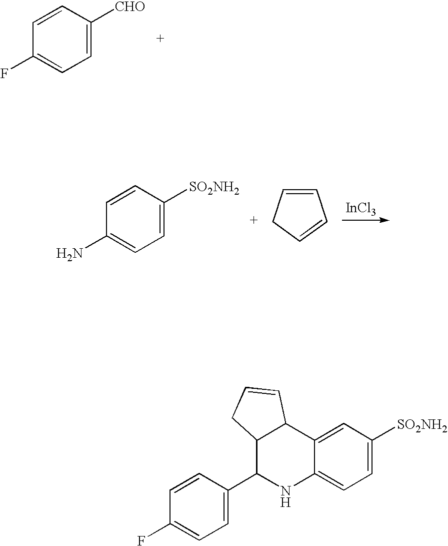

¹H NMR (500 MHz, DMSO-d₆) δ 7.50 (m, 2H), 7.45 (s, 1H), 7.35 (m, 1H), 7.25 (m, 2H), 6.97 (s, 2H), 6.80 (d, 1H), 6.36 (s, 1H), 5.90 (m, 1H), 5.6 (m, 1H), 4.65 (m, 1H), 4.05 (m, 1H), 2.93 (m, 1H), 2.35 (m, 1H), 1.62 (m, 1H); MS (ES+) m/z: 345 (M+1); Anal. Calcd. for C₁₈H₁₇F₁N₂O₂S: C, 62.77; H, 4.98; N, 8.13. Found: C, 62.59; H, 5.42; N, 8.47.

### Example 13

### 4-(4-Methylphenyl)-2,3,3a,4,5,9b-hexahydro-furo[3,2-c]quinoline-8-sulphonamide.

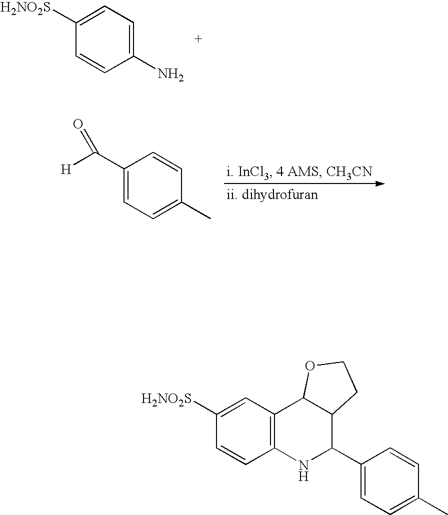

Sulfanilimide (0.47 g, 2.7 mmol), p-tolualdehyde (0.29 ml, 2.5 mmol), indium trichloride (0.11 g, 0.50 mmol), and 4 Å molecular sieves (1.28 g) in dry acetonitrile (3 mL) was stirred at room temperature for 15 min under nitrogen. 2,3-Dihydrofuran (0.83 mL, 11.0 mmol) was then added and the reaction stirred for 48 hours. The mixture was filtered through a silica gel plug using acetonitrile, and the filtrate concentrated. The solid was absorbed onto silica gel and flashed using 1:5 isopropanol-hexane to give a white solid (120 mg, 14%). ¹H NMR (300 MHz, DMSO-d₆): 7.92 (s, 1H), 7.61 (d, 1H, J=8.4 Hz), 7.19-7.33 (m, 7H), 6.62 (d, 1H, J=8.4 Hz), 5.24 (d, 1H, J=7.5 Hz), 4.78 (s, 1H), 4.67 (s, 1H, br), 3.74-3.85 (m, 2H), 2.77 (m, 1H), 2.40 (s, 3H), 1.65-1.80 (m, 1H), 1.55-1.65 (m, 1H). LCMS (ES) 345.3 (M+H).

### Example 14

### (3aR,4S,9bS)-4-Naphthalen-2-yl-3a,4,5,9b-tetrahydro-3H-cyclopenta[c]quinoline-8-sulphonamide and

### Example 15

### (3aS,4R,9bR)-4-Naphthalen-2-yl-3a,4,5,9b-tetrahydro-3H-cyclopenta[c]quinoline-8-sulphonamide.

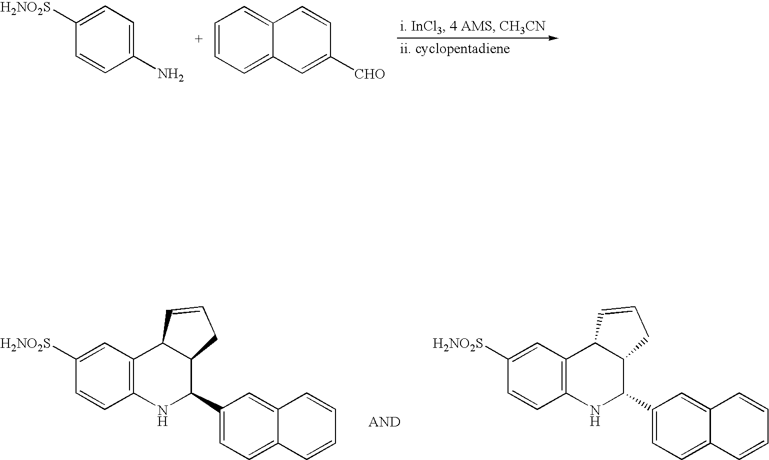

Sulfanilimide (9.1 g, 0.053 mol), 2-naphthaldehyde (7.5 g, 0.048 mol), indium trichloride (3.7 g, 0.017 mol), and 4 Å molecular sieves (10 g) in dry acetonitrile (120 mL) was stirred at room temperature for 15 min under nitrogen. Cyclopentadiene (17.3 mL, 0.21 mol) was then added and the reaction stirred for 3 hours. The reaction mixture was filtered through a silica gel plug, washed with acetonitrile, and the filtrate concentrated. The solid was absorbed onto silica gel and flashed with hexane-isopropanol (10:1) to give a white solid (2.1 g). A portion of the crude material (50 mg) was purified to yield the major pair (minor pair not isolated) using supercritical fluid chromatography on a chiracel OD column with isocratic 50:50 MeOH:CO₂ to give the faster eluting title compound (12 mg, 3%) as an off-white solid. ¹H NMR (300 MHz, DMSO-d₆): 7.92-7.98 (m, 4H), 7.60 (d, 1H, J=8.7 Hz), 7.50-7.53 (m, 2H), 7.47 (s, 1H), 7.36 (d, 1H, J=8.4 Hz), 6.97 (s, 2H), 6.86 (d, 1H, J=8.4 Hz), 6.52 (s, 1H), 5.90 (s, 1H, br), 5.62 (s, 1H, br) 4.81 (s, 1H, br), 4.13 (d, 1H, J=9.0 Hz), 3.08 (m, 1H), 2.43 (m, 1H), 1.62 (dd, 1H, J=9.3, 16.2 Hz). LC/MS (ES) 377.3 (M+H). [α_{D}]=(−). The slower eluting title compound was also isolated as an off-white solid (26 mg, 6%). ¹H NMR (300 MHz, DMSO-d₆): 7.91-7.97 (m, 4H), 7.60 (d, 1H, J=8.4 Hz), 7.50-7.53 (m, 2H), 7.46 (s, 1H), 7.36 (dd, 1H, J=2.1, 8.7 Hz), 6.97 (s, 2H), 6.86 (d, 1H, J=8.4 Hz), 6.52 (s, 1H), 5.90 (s, 1H, br), 5.62 (d, 1H, J=4.8 Hz), 4.81 (d, 1H, J=2.7 Hz), 4.12 (d, 1H, J=9.0 Hz), 3.08 (m, 1H), 2.43 (m, 1H), 1.62 (dd, 1H, J=9.0, 15.6 Hz). LC/MS (ES) 377.1 (M+H). [α_{D}]=(+).

### Example 16

### (3aR,4R,9bS)-4-(4-Methylphenyl)-3a,4,5,9b-tetrahydro-3H-cyclopenta[c]quinoline-8-sulfonamide.

### Example 17

### (3aR,4S,9bS)-4-(4-Methylphenyl)-3a,4,5,9b-tetrahydro-3H-cyclopenta[c]quinoline-8-sulfonamide,

### Example 18

### (3aS,4R,9bR)-4-(4-Methylphenyl)-3a,4,5,9b-tetrahydro-3H-cyclopenta[c]quinoline-8-sulfonamide, and

### Example 19

### (3aS,4S,9bR)-4-(4-Methylphenyl)-3a,4,5,9b-tetrahydro-3H-cyclopenta[c]quinoline-8-sulfonamide.

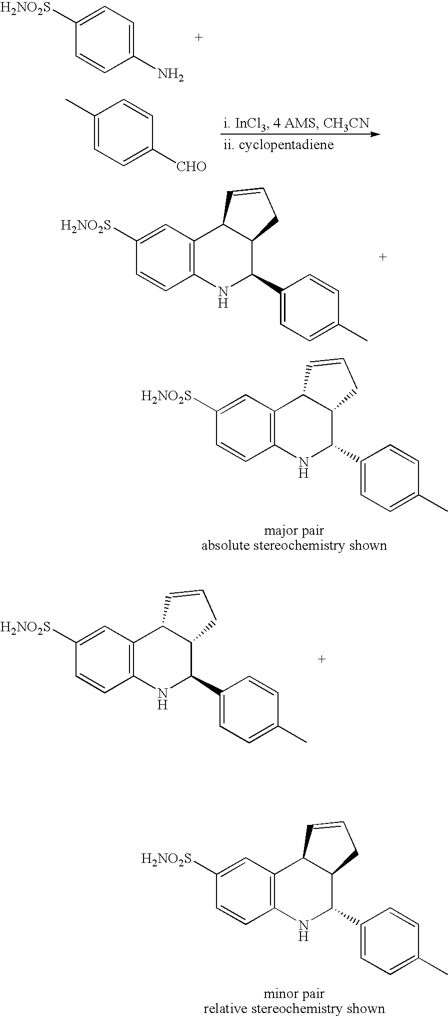

Sulfanilimide (20.5 g, 0.12 mol), p-tolualdehyde (12.7 mL, 0.11 mol), indium trichloride (4.8 g, 0.022 mol), and 4 Å molecular sieves in dry acetonitrile (125 mL) was stirred at room temperature for 15 min under nitrogen. Cyclopentadiene (31.4 mL, 0.48 mol) was then added and the reaction stirred for 48 hours. The mixture was filtered through a silica gel plug, washed with acetonitrile, and the filtrate concentrated. The solid was recrystallized from isopropanol-hexane to give a white solid (4.2 g). A portion of the recrystallized material (150 mg) was submitted to supercritical fluid chromatography on a chiracel OD column using isocratic 35% MeOH in CO₂. Four compounds were isolated, and are designated as Fractions 1-4 based on the order of elution:

Fractions 1 (Example 16) and 3 (Example 19) were assigned as (3aR,4R,9bS)-4-(4-methylphenyl)-3a,4,5,9b-tetrahydro-3H-cyclopenta[c]quinoline-8-sulfonamide, and (3aS,4S,9bR)-4-(4-methylphenyl)-3a,4,5,9b-tetrahydro-3H-cyclopenta[c]quinoline-8-sulfonamide based on NMR spectroscopy and one.

Fraction 1, white solid (6 mg, 0.4%): ¹H NMR (DMSO-d₆) 7.58 (s, 1H), 7.35 (d, 1H, J=8.4 Hz), 7.28 (d, 2H, J=7.8 Hz), 7.20 (d, 2H, J=7.5 Hz), 6.94 (s, 2H), 6.73 (d, 1H, J=8.4 Hz), 6.52 (s, 1H), 5.89 (m, 1H), 5.74 (s, br, 1H), 3.90 (s, br, 1H), 3.59 (d, 1H, J=9.5 Hz), 2.59-2.61 (m, 1H), 2.36-2.4 (m, 1H), 2.31 (s, 3H), 1.99-2.05 (m, 1H). LCMS (ES) 341.3 (M+1).

Fraction 3, white solid (5 mg, 0.4%): ¹H NMR (DMSO-d₆) 7.58 (s, 1H), 7.35 (d, 1H, J=8.4 Hz), 7.28 (d, 2H, J=7.8 Hz), 7.20 (d, 2H, J=7.5 Hz), 6.94 (s, 2H), 6.73 (d, 1H, J=8.4 Hz), 6.52 (s, 1H), 5.89 (m, 1H), 5.74 (s, br, 1H), 3.90 (s, br, 1H), 3.59 (d, 1H, J=9.5 Hz), 2.59-2.61 (m, 1H), 2.36-2.4 (m, 1H), 2.31 (s, 3H), 1.99-2.05 (m, 1H). LCMS (ES) 341.3 (M+1).

Fraction 2 (Example 17 was assigned as (3aR,4S,9bS)-4-(4-methylphenyl)-3a,4,5,9b-tetrahydro-3H-cyclopenta[c]quinoline-8-sulfonamide, based on N spectroscopy and one. The absolute stereochemistry was assigned as (3aR,4S,9bS) based on the comparison of measured and calculated vibrational circular dichroism spectra.

Fraction 2, white solid (45 mg, 3%): ¹H NMR (DMSO-d₆) 7.42 (s, 1H), 7.31-7.34 (m, 3H), 7.19 (d, 2H, J=7.8 Hz), 6.94 (s, 2H), 6.80 (d, 1H, J=8.7 Hz), 6.31 (s, 1H), 5.87 (m, 1H), 5.62 (m, 1H), 4.58 (m, 1H), 4.06 (d, br, 1H, J=8.1 Hz), 2.92 (dd, 1H, J=J=7.2Hz), 2.37-2.42 (m, 1H), 2.31 (s, 3H), 1.64 (dd, 1H, J=7.5, 14.4 Hz). LCMS (ES) 341.3 (M+1), Calc for C₁₉H₂₀N₂O₂S with 0.1 H₂O: C, 65.74; H, 5.83; N, 8.03. Found: C, 65.83; H, 5.62; N 7.86. [α_{D}]=+0.8° (c=0.5, CH₃OH).

Fraction 4 (Example 18) was assigned as (3aS,4R,9bR)-4-(4methylphenyl)-3a,4,5,9b-tetrahydro-3H-cyclopenta[c]quinoline-8-sulfonamide, based on NMR spectroscopy and one. The absolute stereochemistry was assigned as (3aS,4R,9bR) based on the comparison of measured and calculated vibrational circular dichroism spectra.

Fraction 4, white solid (65 mg, 3%): ¹H NMR (DMSO-d₆) 7.42 (s, 1H), 7.31-7.34 (m, 3H), 7.19 (d, 2H, J=7.8 Hz), 6.94 (s, 2H), 6.80 (d, 1H, J=8.7 Hz), 6.31 (s, 1H), 5.87 (m, 1H), 5.62 (m, 1H), 4.58 (d, 1H, J=2.7 Hz), 4.06 (d, br, 1H, J=8.1 Hz), 2.92 (dd, 1H, J=J=7.2 Hz), 2.37-2.42 (m, 1H), 2.31 (s, 3H), 1.64 (dd, 1H, J=7.5, 14.4 Hz). LCMS (ES) 341.3 (M+1). [α_{D}]=−0.8° (c=0.5, CH₃OH).

### Example 20

### (3aR,4S,9bS)-4-(4-Methylphenyl)-1,2,3a,4,5,9b-hexahydro-3H-cyclopenta[c]quinoline-8-sulfonamide

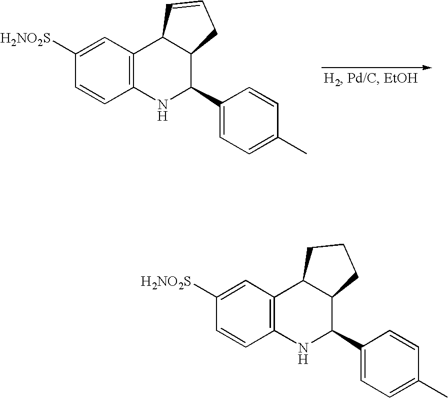

A solution of (3aR,4S,9bS)-4-(4-Methylphenyl)-3a,4,5,9b-tetrahydro-3H-cyclopenta[c]quinoline-8-sulfonamide (Example 17, 269 mg, 0.79 mmnol) in 10 mL of THF was added to a suspension of palladium on carbon (10%, 42 mg, 0.04 mmol, 5 mol %) in 10 mL of absolute ethanol in a 100 mL Paar flask. The resulting mixture was shaken for 1 hour on a Paar hydrogenator under a hydrogen atmosphere (50 psi) then filtered through a pad of diatomaceous earth. The filtrate was concentrated under vacuum (10 torr) to give the title compound as a white solid. Yield: 262 mg (97%). 1H NMR: (CDCl₃, 600 MHz) d: 7.68 (s, 1H), 7.51 (d, 1H, J=8.6 Hz), 7.27 (d, 2H, J=7.9 Hz), 7.17 (d, 2H, J=7.6 Hz), 6.59 (d, 1H, J=3 8.3 Hz), 4.64 (m, 1H), 4.60 (br s, 2H, NH₂), 4.29 (br s, 1H, NH), 3.45 (dd, 1 H, J₁=7.6 Hz, J₂=7.2 Hz), 2.48-2.43 (m, 1H), 2.36 (s, 3 H), 2.20-2.14 (m, 1H), 1.93-1.86 (m, 1H), 1.66-1.60 (m, 1H), 1.51-1.45 (m, 1H), 1.32-1.27 (m, 1H); MS (APCI) M+H 343.

### Example 21

### (3aS,4R,9bR)-4-(4-Methylphenyl)-1,2,3a,4,5,9b-hexahydro-3H-cyclopenta[c]quinoline-8-sulfonamide

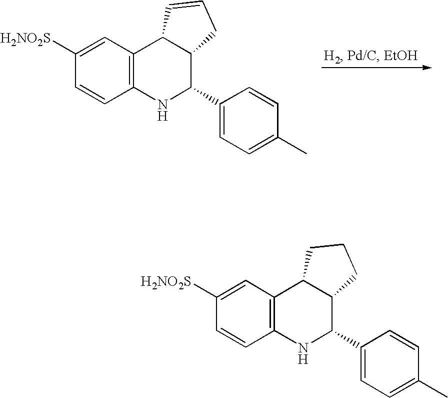

A solution of (3aS,4R,9bR)-4-(4-Methylphenyl)-3a,4,5,9b-tetrahydro-3H-cyclopenta[c]quinotine-8-sulfonamide (Example 18, 340 mg, 1.0 mmol) in 10 mL of THF was added to a suspension of palladium on carbon (10%, 42 mg, 0.04 mmol) in 10 mL of absolute ethanol in a 100 mL Paar flask. The resulting mixture was shaken for 1 hour on a Paar hydrogenator under a hydrogen atmosphere (50 psi) then filtered through a pad of diatomaceous earth. The filtrate was concentrated under vacuum (10 torr) to give the title compound as a white solid. Yield: 297 mg (86%). 1H NMR: (CDCl₃, 300 MHz) d: 7.68 (s, 1H), 7.51 (dd, 1H, J₁=8.6 Hz, J₂=2.2 Hz), 7.27 (d, 2H, J=7.9 Hz), 7.17 (d, 2H, J=7.9 Hz), 6.59 (d, 1H, J=8.8 Hz), 4.64 (m, 1H), 4.60 (br s, 2H, NH₂), 4.29 (br s, 1H, NH), 3.45 (dd, 1H, J₁=7.6 Hz, J₂=7.2 Hz), 2.48-2.43 (m, 1H), 2.36 (s, 3 H, 2.20-2.14 (m, 1H), 1.93-1.86 (m, 1H), 1.66-1.60 (m, 1H), 1.51-1.45 (m, 1H), 1.32-1.27 (m, 1H); MS (APCI) M+H 343.

1. A method of treatment or prophylaxis of psychotic disorders, intellectual impairment disorders or diseases or conditions in which modulation of the α7 nicotinic receptor is beneficial, which method comprises administering a therapeutically-effective amount of a compound of Formula I or formula II: 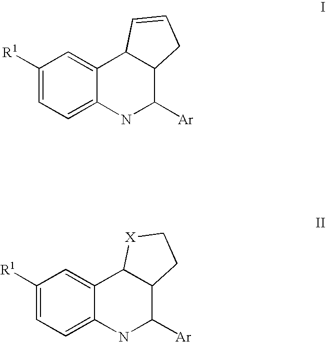
wherein:
R¹ is —OH, —N(R²)₂, —NR²—SO₂—R², —SO₂—N(R²)₂, —CON(R²)₂, or —NR²COR² where R² at each occurrence is independently selected from hydrogen, C₁₋₄alkyl, halogenatedC₁₋₄alkyl, aryl or heteroaryl where any alkyl, halogenated-alkyl, aryl or heteroaryl moiety is substituted with 0, 1, 2 or 3 R³ moieties;
X is O, S or CH₂;
Ar is a moiety selected from furyl, pyridyl, thienyl, phenyl or naphthyl, said moiety having 0, 1, 2, 3 or more R³ substituents where R³ is at each occurrence independently selected from hydrogen, halogen, C₁₋₄alkyl, C₂₋₄alkenyl, C₂₋₄alkynyl, OC₁₋₄alkyl, NH₂, CO₂H, CO₂C₁₋₄alklyl, CN, NO₂, and CF₃;
or a diastereoisomer, enantiomer or pharmnaceutically-acceptable salt thereof.

2. A method of treatment or prophylaxis according to claim 1, wherein said psychotic disorder, intellectual impairment disorder or disease or condition in which modulation of the α7 nicotinic receptor is beneficial is selected from Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Hluntington's disease, Tourette's syndrome, a neurodegenerative disorder in which there is loss of cholinergic synapse, jetlag, nicotine addiction, pain, ulcerative colitis or irritable bowel syndrome.

3. A pharmaceutical composition comprising a compound according to Formula I or Formula II 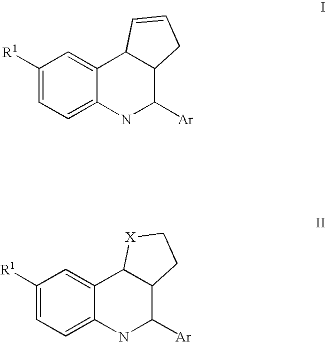
wherein:
R¹ is —OH, —N(R²)₂, —NR²—SO₂—R², —SO₂—N(R²)₂, —CON(R²)₂, or —NR²COR² where R² at each occurrence is independently selected from hydrogen, C₁₋₄alkyl, halogenatedC₁₋₄alkyl, aryl or heteroaryl where any alkyl, halogenated-alkyl, aryl or heteroaryl moiety is substituted with 0, 1, 2 or 3 R³ moieties;
X is O, S or CH₂;
Ar is a moiety selected from furyl, pyridyl, thienyl, phenyl or naphthyl, said moiety having 0, 1, 2, 3 or more R³ substituents where R³ is at each occurrence independently selected from hydrogen, halogen, C₁₋₄akyl, C₂₋₄alkenyl, C₂₋₄alkynyl, OC₁₋₄alkyl, NH₂, CO₂H, CO₂C₁₋₄alkyl, CN, NO₂, and CF₃;
or a diastereoisomer, enantiomner or pharmaceutically-acceptable salt thereof, together with at least one pharmaceutically-acceptable diluent or carrier.

4. The pharmaceutical composition according to claim 3, in addition comprising a nicotinic receptor agonist.

5. A method of treatment prophylaxis of Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, ulcerative colitis or irritable bowel syndrome comprising administering a therapeutically-effective amount of a pharmaceutical composition according to claim 3.

6. (canceled)

7. A compound of Formula I or Formula II: 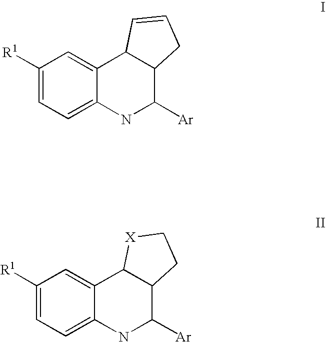
wherein:
R¹ is NR²—SO₂—R² or —SO₂—N(R²)₂ where R² at each occurrence is independently selected from hydrogen, C₁₋₄alkyl, halogenatedC₁₋₄alkyl, aryl or heteroaryl where any alkyl, halogenated-alkyl, aryl or heteroaryl moiety is substituted with 0, 1, 2 or 3 R³ moieties;
X is O, S or CH₂;
Ar is a moiety selected from furyl, pyridyl, thienyl, phenyl or naphthyl, said moiety having 0, 1, 2, 3 or more R³ substituents where R³ is at each occunence independently selected from hydrogen, halogen, C₁₋₄alkyl, C₂₋₄alkenyl, C₂₋₄alkynyl, OC₁₋₄alkyl, NH₂, CO₂H, CO₂C₁₋₄aklkyl, CN, NO₂, and CF₃;
or a diastereoisomer, enantiomer or pharmaceutically-acceptable salt thereof.

8. A compound according to claim 7, wherein:
R¹ is —SO₂—N(R²)₂ where R² at each occurrence is independently selected from hydrogen, C₁₋₄alkyl, halogenatedC₁₋₄alkyl, aryl or heteroaryl where any alkyl, halogenated-alkyl, aryl or heteroaryl moiety is substituted with 0, 1, 2 or 3 R³ moieties;
X is O, S or CH₂;
Ar is a moiety selected from furyl, pyridyl, thienyl, phenyl or naphthyl, said moiety having 0, 1, 2, 3 or more R³ substituents where R³ is at each occurrence independently selected from hydrogen, halogen, C₁₋₄alkyl, C₂₋₄alkenyl, C₂₋₄alkynyl, OC₁₋₄alkyl, NH₂, CO₂H, CO₂C₁₋₄alkyl, CN, NO₂, and CF₃;

9. A compound according to claim 7, said compound being:
4-(2-methylphenyl)-3a,4,5,9b-tetrahydro-3H-cyclopenta[c]quinoline-8-sulfonamide;
4-(4-methylphenyl)-3a,4,5,9b-tetrahydro-3H-cyclopenta[c]quinoline-8-sulfonamide;
4-(3,4,5-trimethoxyphenyl)-3a,4,5,9b-tetrahydro-3H-cyclopenta[c]quinoline-8-sulfonamide;
4-(2-methyl-4,5-dimethoxyphenyl)-3a,4,5,9b-tetrahydro-3H-cyclopenta[c]quinoline-8-sulfonamide;
4-(3,5-dimethoxyphenyl)-3a,4,5,9b-tetrahydro-3H-cyclopenta[c]quinoline-8-sulfonamide;
4-(4-tert-butylphenyl)-3a,4,5,9b-tetrahydro-3H-cyclopenta[c]quinoline-8-sulfonamide;
4-(2-naphthyl)-3a,4,5,9b-tetrahydro-3H-cyclopenta[c]quinoline-8-sulfonamide;
4-(4fluorophenyl)3a,4,5,9b-tetrahydro-3H-cyclopenta[c]quinoline-8-sulfonamide;
8-methyl-4-(4-methylphenyl)-2,3,3a,4,5,9b-hexahydro-furo[3,2-c]quinoline;
(3aR,4S ,9bS)-8-methyl-4-naphthalen-2-yl-3a,4,5,9b-tetrahydro-3H-cyclopenta[c]quinoline;
(3aS,4R,9bR)-8-methyl-4-naphthalen-2-yl-3a,4,5,9b-tetrahydro-3H-cyclopenta[c]quinoline;
(3aR,4S ,9bS)-4-(4-methylphenyl)-3a,4,5,9b-tetrahydro-3H-cyclopenta[c]quinoline-8-sulfonamide;
(3aS,4R,9bR)-8-methyl-4-(4-methylphonyl)-3a,4,5,9b-tetrahydro-3H-cyclopenta[c]quinoline-8-sulfonamide;
(3aS,4S,9bR)-4-(4-methylphenyl)-3a,4,5,9b-tetrahydro-3H-cyclopenta[c]quinoline-8-sulfonamide;
(3aR,4R,9bS)-4-(4-methylphenyl)-3a,4,5,9b-tetrahydro-3H-cyclopenta[c]quinoline-8-sulfonamide;
(3aR,4S,9bS)-4-(4-methylphenyl)-1,2,3a,4,5,9b-hexahydro-3H-cyclopenta[c]quinoline-8-sulfonamide or
(3aS,4R,9bR)-4-(4-methylphenyl)-1,2,3a,4,5,9b-hexahydro-3H-cyclopenta[c]quinoline-8 -sulfonarnide
or a phamaceutically-acceptable salt thereof.

10. A method of making a compound according to Formula I or Formula II 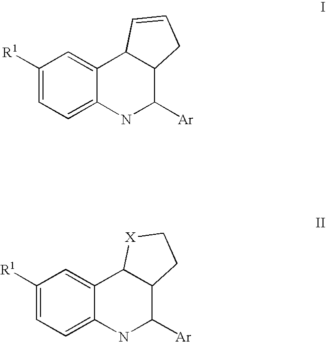
wherein:
R¹ is NR²—SO₂—R² or —SO₂—N(R²)₂ where R² at each occurrence is independently selected from hydrogen, C₁₋₄alkyl, halogenatedC₁₋₄alkyl, aryl or heteroaryl where any alkyl, halogenated-alkyl, aryl or heteroaryl moiety is substituted with 0, 1, 2 or 3 R³ moieties;
X is O, S or CH₂;
Ar is a moiety selected from furyl, pyridyl, thienyl, phenyl or naphthyl, said moiety having 0, 1, 2, 3 or more R³ substituents where R³ is at each occurrence independently selected from hydrogen, halogen, C₁₋₄alkyl, C₂₋₄alkenyl, C₂₋₄alkynyl, OC₁₋₄alkyl, NH₂, CO₂H, CO₂C₁₋₄alkyl, CN, NO₂, and CF₃; comprising:
adding indium chloride to a solution of an arylaldehyde of formula Ar—CHO.
a 4-aminobenzenesulfonamide of the following formula 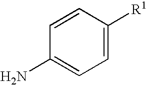
and cyclopentadiene or a compound of the following formula 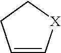
in acetonitrile and stirring overnight;
neutralizing, extracting, concentrating and purifying to afford a quinoline of Formula I or Formula II.
